(19) United States
(12) Patent Application Publication
(10) Pub. No.: US 2007/0017548 A1
(43) Pub. Date: Jan. 25, 2007

(54) System and method for cleaning or sanitizing items intended for re-use

(76) Inventors: Tobin R. King, Carrollton, GA (US); Albert Christopher Allen, Sharpsburg, GA (US)

Correspondence Address: MICHAEL E. McKEE;Attorney at Law, 804 Swaps Lane, Knoxville, TN, 37923, US

(21) Appl. No.: 11/510,895

(22) Filed: Aug. 28, 2006

Related U.S. Application Data
(63) Continuation of application No. 11/183,539, filed on Jul. 18, 2005.

(51) Int. Cl. B08B 3/12; B08B 7/00; B08B 3/00; B08B 6/00

(52) U.S. Cl. 134/1; 134/123; 134/137; 134/199; 134/103.1; 134/33; 134/34

(57) ABSTRACT

A system and method for sanitizing an item, such as a grocery store shopping cart or a child stroller, for re-use utilizes a passageway having an entrance and an exit and a source of ozinated water. A pump and an associated piping network are used to direct the water, when in an ozinated condition, from the source toward the item so that the surfaces of the item which are struck by the ozinated water are sanitized thereby. The water can be collected after it has been directed toward the item for subsequent use in the system.

This is a continuation of application Ser. No. 11/183,539, filed Jul. 18, 2005 and bearing the same title as this application, and the disclosure of which is incorporated herein by reference.

### BACKGROUND OF THE INVENTION

This invention relates generally to the cleaning or sanitizing of surfaces and relates, more particularly, to means and methods for sanitizing items which are commonly exposed to bacteria yet are intended to be used repeatedly.

The class of items with which this invention is concerned includes shopping carts and child strollers, such as those which are employed by individuals for either collecting food items within a grocery store for purchase or for transporting children through a store or theme park. Such items are also expected to be re-used by others after use.

Items of this class includes surfaces which, during use, might be exposed to undesirable material, such as the drippings from raw chicken and other meats or dirty diapers worn by children who sit within such an item, or might be contacted by the unwashed hands of an individual who uses such an item while he is sick. If left uncleaned after use, the exposed surfaces of such an item could spawn the growth of unwanted micro-cultures, such as salmonella, e coli, botulism and enteroccocus facaelis, to which a subsequent user of the item could be exposed.

With regard to grocery store shopping carts, efforts have been made to clean the carts with a steam wand in the store parking lot, but any bacteria which is washed from the cart surfaces, if left untreated, could migrate into and pollute a public water source (e.g. a river, lake, stream or ocean). Some grocery stores have made sanitized wipes available which permit a customer to manually wipe a shopping cart clean before it is used, but the amount of time and effort normally required to adequately clean a shopping cart in this manner often discourages the customer from cleaning the shopping cart at all.

It would therefore be desirable to provide an improved means and an associated method for sanitizing or cleaning an item of the aforedescribed class which requires relatively little time and effort to operate or carry out and which reduces the likelihood that harmful bacteria which could be spawned upon the container surfaces would migrate to the public water supply following a sanitizing or cleaning operation.

Accordingly, it is an object of the present invention to provide a new and improved system and method for sanitizing or cleaning a food-collection container, such as a grocery store shopping cart or a child stroller.

Another object of the present invention is to provide such a system which does not require that the surfaces of the item be manually rubbed or wiped during the sanitizing process.

Still another object of the present invention is to provide such a system whose operation is automatic and requires relatively little time or effort to sanitize and clean an item.

A further object of the present invention is to provide such a system which is particularly well-suited for sanitizing or cleaning grocery store shopping carts and child strollers.

A still further object of the present invention is to provide such a system which is uncomplicated in structure, yet effective in operation.

### SUMMARY OF THE INVENTION

This invention resides in a system and method for sanitizing an item, such as a grocery store shopping cart or a stroller, for re-use.

The system includes a source of ozinated water and means for directing the ozinated water from the source toward the item so that the surfaces of the item which are exposed to the ozinated water are sanitized thereby.

The method of the invention includes the steps performed by the system of the invention. In particular, such steps include the providing a source of ozinated water, and directing the ozinated water from the source toward the item to sanitize the surfaces of the item.

### BRIEF DESCRIPTION OF THE DRAWINGS

FIG. 1 is a perspective view, shown partially cut-away, of an embodiment of a cleaning and sanitizing system within which features of the present invention are embodied.

FIG. 2 is a side elevational view, shown partially cut-away, of the FIG. 1 system and a plurality of shopping carts being advanced through the system.

FIG. 3 is a cross-sectional view of a fragment of the FIG. 1 embodiment taken along line 3-3 of FIG. 1.

FIG. 4 is a cross-sectional view of a fragment of the FIG. 1 embodiment taken along line 4-4 of FIG. 1.

FIG. 5 is a view which schematically illustrates in block diagram form the operation of several components of the FIG. 1 system.

### DETAILED DESCRIPTION OF AN ILLUSTRATIVE EMBODIMENT

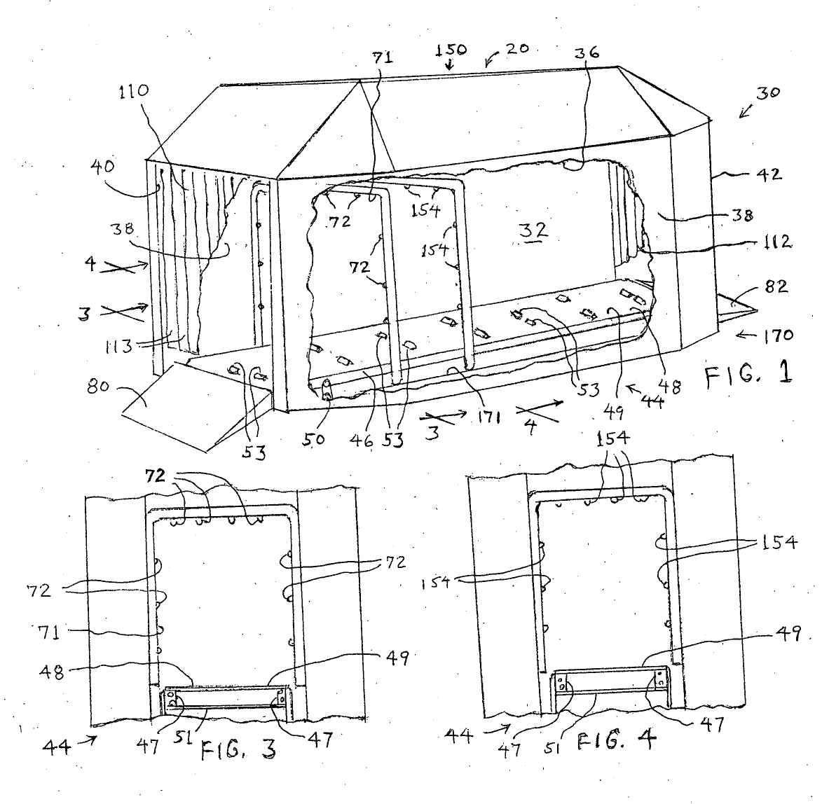

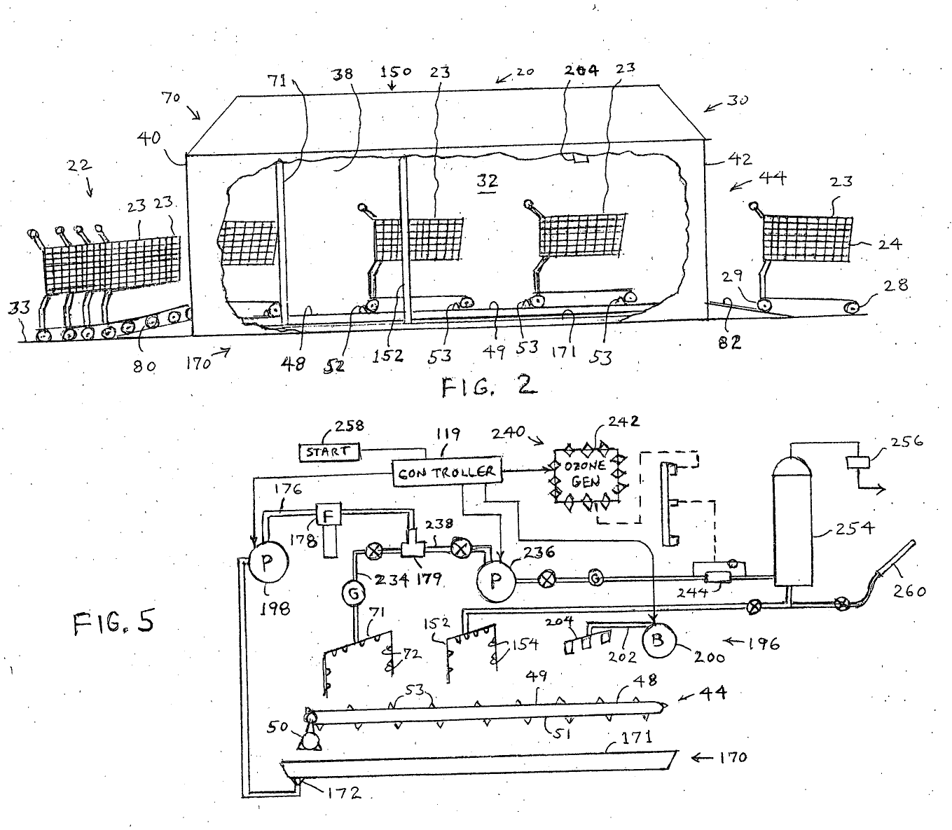

Turning now to the drawings in greater detail and considering first FIGS. 1 and 2, there is illustrated an embodiment, generally indicated 20, of a sanitizing system within which features of the present invention are embodied and which can be used to clean and sanitize the surfaces of items 22 as the items 22 are advanced through the system 20. The item 22 intended to be cleaned and sanitized with the depicted system 20 is a shopping cart 23 which is commonly used by a grocery store customer to collect food items for purchase. As is common with grocery store shopping carts and as best shown in FIG. 2, each shopping cart 23 includes front and rear wheels 28 and 29, respectively, and a container section 24 having a bottom and sidewalls comprised of grillwork which can be constructed of metal, plastic or other suitable material.

During use of the shopping cart 23, undesirable materials might be transferred to the surfaces of the container section 24 by selected items of food or by the customer or his children. For example, during use of the shopping cart 23, its surfaces might be exposed to the drippings from raw chicken and other meats or even to the dirty diapers worn by children who are permitted sit within the shopping cart 23. Further still, the shopping cart surfaces might be contacted by the unwashed hands of a customer who uses the shopping cart 23 while he is sick. Of course, if such surfaces of the shopping cart 23 are left uncleaned after use, any unwanted material transferred thereto could spawn the growth of unwanted micro-cultures, such as salmonella, e coli, botulism and enteroccocus facaelis, to which a subsequent user of the shopping cart 23 could be exposed.

As will be apparent herein, the system 20 can be used to quickly and effectively clean and sanitize the surfaces of the depicted shopping cart 23 after each use so that the depicted shopping cart 23 is relatively free of active undesirable microorganisms for use by a subsequent customer. In this connection, the system 20 employs a washing (e.g. a cleaning) stage, a sanitizing stage, and a drying stage, and the operation of the various components (described herein) of the system 20 are controlled by a computer controller 119 (FIG. 5) so that once the operation of the system 20 is initiated, little, if any, operator intervention is necessary.

With reference again to FIGS. 1 and 2, the system 20 includes means, generally indicated 30, defining an elongated passageway 32 through which a plurality of shopping carts 23 are automatically moved in preparation of and upon completion of a sanitizing operation performed with the system 20. The passageway-defining means 30 of the depicted system 20 includes a top 36 and two opposite sidewalls 38 which extend between the top 36 and the floor 33 or similar underlying support surface. One end, indicated 40, of the passageway 32 provides an entrance for the passageway-defining means 30, and the opposite end, designated 42, of the passageway 32 provides an exit for the passageway-defining means 30. As will be apparent herein, shopping carts 23 which are desired to be sanitized within the system 20 are moved in an end-to-end fashion into the passageway 32 at the entrance 40 thereof, and upon completion of the cleaning and sanitizing operation, the shopping carts 23 exit the passageway 22 at the exit 42 thereof.

With reference to FIGS. 1-4, the passageway-defining means 30 includes an entrance curtain 110 (FIG. 1) adjacent the passageway entrance 40 and includes an exit curtain 112 (FIG. 1) adjacent the passageway exit 42. Each curtain 110 and 112 includes a plurality of flexible plastic strips 113 which are suspended from the top 36 of the passageway-defining means 30 and which collectively form a closure at the corresponding end 40 or 42 through which the carts 23 are permitted to pass without damaging the curtain 110 or 112. In practice, as each cart 23 is conveyed into contact with the curtain 110 or 112, the plastic strips 113 are spread apart by the front of the cart 23 so that as the cart 23 continues to advance through the curtain 110 or 112, the strips 113 are permitted to slide along the sides of the cart 23. As each cart 23 passes through the curtain 110 or 112, the strips 113 return to a closure-forming condition under the influence of gravity.

For purposes of moving the shopping carts 23 through the passageway 32, the system 20 includes conveyor means, generally indicted 44 in FIGS. 1 and 2, for conveying the shopping carts 23 through the passageway 32 from the entrance end 40 to the exit end 42 thereof. In this connection, the conveyor means 44 includes an elongated frame 46 which extends between the ends 40 and 42 of the passageway 32 and sprockets 47 (FIGS. 3 and 4) which are rotatably mounted along each side of the support surface adjacent the passageway ends 40, 42. An endless conveyor 48 (constructed, for example, of a plastic mesh) is looped about the sprockets 47 disposed along each side of the support surface 43 so that an upper section, indicated 49, of the conveyor 48 is movable in one linear direction (e.g. toward the right as illustrated in FIGS. 1 and 2) relative to the frame 46 while a lower section, indicated 51 in FIGS. 3 and 4, of the conveyor 48 is movable in the opposite linear direction (e.g. toward the left as illustrated in FIG. 1). Preferably, a stationary support surface is positioned beneath so as to support the upper section 49 of the conveyor 48 as the upper section 49 is slidably moved therealong. In addition, a plurality of pairs of wheel-engaging cleats 53 are connected to the conveyor 48 so as to project upwardly from the upper section 49 as the upper section 49 is moved between the passageway ends 40 and 42.

For advancing the upper section 49 of the conveyor 48 from the passageway entrance 40 to the passageway exit 42, an electrically-powered motor 50 (FIGS. 1 and 5) is mounted at the frame end corresponding with the passageway entrance 40 and is connected in driving relationship with the sprockets 47 disposed adjacent the exit 42 of the passageway 32 whose teeth are, in turn, operatively connected (e.g. meshed) with the conveyor 48. Therefore, when the motor 50 is energized, the upper section 49 of the conveyor 48 is moved from the entrance end 40 of the passageway 32 toward the exit end 42 thereof while the cleats 53 are moved with the upper section 49.

When a cart 23 is moved (e.g. pushed) onto the upper section 49 of the conveyor 48 adjacent the passageway entrance 40 and the conveyor section 49 is advanced from the passageway entrance 40 toward the passageway exit 42, a pair of cleats 53 carried by the conveyor 48 (and whose spaced-apart distance corresponds with the spaced distance between the two front wheels 28 of the cart 23) abut, or engage, the back of the front wheels 28 so that the cart 23 is advanced, or pushed, through the passageway 32 with the conveyor section 49. If desired, another, or alternative, pair of cleats 53 whose spaced-apart distance corresponds with the spaced distance between the two rear wheels 29 of the cart 23 can be used to engage the back of the rear wheels 29 of the cart 23 for pushing the cart 23 along the support surface 43 toward the passageway exit 42.

To facilitate the advancement of the carts 23 onto and off of the ends of the upper section 49 of the conveyor 48, there is positioned at each end of the conveyor section 49 a ramp section 80 or 82.

It follows that as each shopping cart 23 is directed (e.g. pushed) onto the upper section 49 of the conveyor 48 adjacent the passageway entrance end 40 and the conveyor motor 50 is energized, each appropriately-spaced pair of cleats 53 which are carried by the conveyor 40 are advanced along the passageway 32 with the upper section 49 and into abutting relationship with the back of the front wheels 28 (or the rear wheels 29) of a corresponding cart 23 so that the entire cart 23 is advanced through the passageway 32 with the upper section 29. It also follows that the conveyor means 44 is of sufficient size and strength to suitably transport shopping carts 23 which are placed upon the upper section 49 along the length thereof, and that the spacing between adjacent shopping carts 23 conveyed through the passageway 32 can be controlled by spacing the pair of cleats 53 associated with the conveyor 48 by an appropriate, or preselected, distance apart as measured along the length of the conveyor 48 or, more specifically, the length of the upper section 49.

As mentioned earlier, the carts 23 which are conveyed through the passageway 32 undergo in sequence, a washing (or cleaning) stage, a sanitizing stage, and a drying stage. In this connection and with reference again to FIG. 2, there is provided between the passageway entrance 40 and exit 42 three workstation regions through which a cart 23 is conveyed and through which the three stages (i.e. the washing, sanitizing and drying stages) of operation are carried out upon the cart 23. Within the depicted system 20, the passageway 32 possesses such a size and length to accommodate at least three carts 23 (as illustrated in FIG. 2) positioned therein in an end-to-end arrangement. It will be understood, however, that a system embodiment whose passageway accommodates less than three carts or more than three carts positioned therein can be had. Accordingly, the principles of the present invention can be variously applied.

With reference to FIGS. 1-3, the system 20 includes means, generally indicated 70, for directing, or spraying, water toward the shopping carts 23 as the carts 23 are conveyed through the passageway 32. Such a washing operation is performed upon each cart 23 soon after the cart 23 enters the passageway 32 and is positioned wholly upon the upper section 49 of the conveyor 48. In practice, the water which is directed toward a shopping cart 23 with the directing means 70 is intended to knock solid debris which may be clinging to the cart surfaces from a previous use of the cart 23 in preparation of the subsequent sanitizing stage. Accordingly, the water is directed toward the cart surfaces with sufficient force to accomplish this purpose.

In the depicted embodiment 20, the directing means 70 includes a U-shaped manifold 71 for receiving water from a source and a series of turbo nozzles 72 (e.g. ten) which are mounted within the legs and base of the manifold 71. The manifold 71 is, in turn, supported within the passageway 32 so that the nozzles 72 are arranged across the top and sides of the passageway 32 and which are directed inwardly of the interior of the passageway 32 so that carts 23 which are conveyed through (the first workstation region of) the passageway 32 are struck by the water which is directed out of the nozzles 72. In use, each turbo nozzle 72 imparts a swirling motion to the water stream which exits the nozzle 72 to thereby enhance the cleaning action of the water which strikes the cart surfaces. It has been found that the surfaces of the carts 23 can be satisfactorily washed with only a few moments of exposure to the water directed from the nozzles 72.

For delivering the water from a source to the nozzles 72, the directing means 70 includes a centrifugal pump 198 (FIG. 5) disposed between the nozzles 72 and the source of the water.

Upon completion of the washing stage of the system operation (during which the surfaces of the carts 23 are exposed to the water directed from the nozzles 72), the carts 23 are conveyed into the next, i.e. second. workstation region of the passageway 32 where the carts are sanitized in a sanitizing operation. In this connection, the system 20 includes sanitizing means, generally indicated 150, for directing a spray of ozinated water toward the shopping carts 23 while the carts 23 are conveyed through the workstation region disposed downstream of the manifold 71. In this connection and as best shown in FIG. 4, the ozinated water-directing means 150 includes a U-shaped spray manifold 152 (FIGS. 1 and 4) which is positioned within the passageway 32 so that the legs and base of the U of the manifold 152 extend upwardly along the sides of and across the top of the carts 23 which are conveyed through the passageway 32. The spray manifold 152 includes a plurality of (e.g. ten) turbo nozzles 154 which are directed toward the interior of the station 62 so that the ozinated water which is delivered to the nozzles 154 in a pressurized condition (e.g. between about 30 and 55 psi) is directed out of the nozzles 154 and sprayed over the surfaces of the carts 23 conveyed through the passageway 32. Upon exposure of the surfaces of the carts 23 to the ozinated water, the surfaces of the cart 23 are sanitized.

It has been found that surfaces are sanitized when contacted by ozinated water [i.e. water within which an amount of ozone (O₃) has been dissolved]. While the dissolved ozone has been known to escape the water within which the ozone has been dissolved after the ozinated water is exposed to the atmosphere, the ozone does not escape the ozinated water directed out of the nozzles 154 before the water engages and effectively sanitizes the surfaces of the carts 23. In other words, it is believed that during the use of the system 20, the surfaces of the carts 23 are sanitized within only a few moments of exposure to the ozinated water.

For purposes of conserving the water which is directed over the carts 23 during the washing and sanitizing stages of system operation, the system 20 includes a collection system, generally indicated 170 in FIGS. 1 and 5, which collects water for re-circulation back through the system 20 where it can be re-directed toward the carts 23 being conveyed through the passageway 32 by way of either the nozzles 72 or the nozzles 154. Within the system 20, the collection system 170 includes a collection, or sump, tray 171 positioned beneath the conveyor 48 for gravitationally collecting any water which has been directed toward the carts 23. Once collected within the tray 171, the water is permitted to gravitationally flow toward a drain 172 (FIG. 5) disposed at the bottom of the tray 171. From the drain 172, the liquid medium is pumped (by means of the pump 198) along a conduit arrangement 176 which includes an internal (e.g. micron) filter 178 for collecting contaminants which may have been dislodged and rinsed from the carts 23. Downstream of the filter 178 is a tee connection 179 for routing the water which is pumped by the pump 198 through one (i.e. a wash stage) conduit network 234 to the turbo nozzles 72 (where it is re-directed over the carts 23 in a washing stage) and through another (i.e. sanitizing stage) conduit network 238 where it is re-ozinated by an ozinating system, generally indicated 240, before the water (in a re-ozinated condition) is re-directed over the carts 23 by way of the turbo nozzles 154 in a sanitizing stage. An optional boost pump 236 can be mounted in the conduit network 238 for aiding the flow of water through the network 238 en route to the turbo nozzles 154.

Associated with the ozinating system 240 is an ozone generator 242 and an ozone injector 244. Briefly, the ozone generator 242 generates ozone gas, and the injector 244 creates a vacuum required to draw the ozone gas from the generator 242 and produces a means by which the generated ozone gas is dissolved in the water flowing through the conduit network 238. During use, the injector 244 produces a cavitation effect, enabling the ozone gas to join the water stream in the form of extremely tiny bubbles. These bubbles must be very small in order to increase the ratio of bubble surface area to the amount of ozone entering the water.

In addition, the ozinating system 240 includes a contact vessel 254 into which ozinated water (or re-ozinated water, as the case may be) is routed which maintains a desired ozone residual within the water. Associated with the contact vessel 254 is an off gas vent 256. As water is directed toward the carts 23 by way of the nozzles 154, the ozinated (or re-ozinated) water is drawn from the base of the contact vessel 254.

For purposes of sanitizing an item (e.g. a shopping cart 23 or baby stroller) with a stream of ozinated water outside of the passageway 32, the system 20 includes a manually-held spray wand 260 which is connected to the contact vessel 254 for receiving ozinated water therefrom. Thus, the spray wand 260 enables a user to sanitize an item of a class which may not be suited for conveying through the passageway 32 or has surface areas desired to be sanitized but may be hard to reach with the water streams delivered from the nozzles 154.

It will be understood that upon collection of the water within the collection tray 171 after it has been directed toward the carts 23, much of the ozone that has been previously dissolved within the water (for purposes of being directed toward the carts 23 through the nozzles 154) escapes from the water. Consequently, by the time that the collected water is pumped from the tray 171 by the pump 198 for delivery through the conduit arrangement 176, the water has returned to a substantially unozinated condition. Therefore and before the water is re-directed toward the carts 23 in an ozinated condition, it must be re-ozinated by means of the ozinating system 240.

An example of a ozinating system, complete with ozone generator, ozone injector, and contact vessel, which has found to be suitable for use as the ozinating system 240 is commercially available from ClearWater Tech, LLC under the trade designation CD10 Series.

Upon completion of the sanitizing stage (during which the cart surfaces are sprayed with ozinated water directed out of the turbo nozzles 154, the carts 23 are exposed to air blown toward the carts 23 in a drying stage. In this connection, the system 20 includes air-directing means, generally indicated 196 in FIG. 5, including a centrifugal blower 200 associated with the passageway-defining means 30 and an air passageway 202 which extends between the exhaust of the blower 200 and terminates in an air manifold 204 mounted in the top of the passageway 32 (and downstream of the manifold 152) so that upon actuation of the blower 200, air is delivered onto the surface of the carts 23 through the passageway 202 to blow water from the surfaces of the carts 23. During operation of the system 20 and, in particular, during the drying stage thereof, air is directed out of the air manifold 204 by the blower 200 and is directed toward the carts 23 which are conveyed through the passageway 32.

A blower which has been found to be well-suited for use as the blower 200 is commercially available from JETAIR Technologies, LLC of Ventura, Calif. under the trade designation JET-2 Centrifugal Blower.

If desired, a curtain comprised of flexible plastic strips (like the curtain 110 or 112 located at the passageway entrance or exit) can be suspended from the top of the passageway to physically separate the passageway region in which a cart 23 is sanitized (i.e. with ozinated water delivered through the nozzles 154) from the adjacent passageway region in which a cart 23 is dried with air from the blower 200. Such a curtain may be advantageous to reduce any likelihood that water which is either directed out of the nozzles 72 or 154 will be blown out of the passageway 32 through the entrance end 40 thereof.

With reference still to FIG. 5, the computer controller 119, introduced earlier, is connected to each of the principal components of the system 20 for controlling the operation of the various system components. Basically, however, once a cart 23 is pushed into the passageway 32 and onto the upper section 49 of the conveyor 48 and a start button 258 is pushed to thereby actuate the conveyor motor 50, the pump 198, the ozone generator 242 and the blower motor 200, operation of the system 20 is automatic. More specifically and as a cart 23 is conveyed by the conveyor 48 through the passageway 32, the cart 32 is exposed first to washing water directed thereat through the nozzles 72, then to the ozinated water directed thereat through the nozzles 154, and finally to the air directed thereat by the blower 200.

It follows from the foregoing that a system 20 and method have been described for washing and sanitizing at least one grocery store shopping cart 23 for re-use. The sanitizing of the shopping cart surfaces is effected with ozinated water which is directed upon the shopping carts 23 as they are conveyed through the passageway 32 for the purpose of killing the bacteria exposed to the ozinated water. Furthermore and because of the dual conduit networks 234 and 238 which utilize water from a common source (i.e. the collection tray 171) and route, through one conduit network 234, unozinated water for washing the carts 23 and route, through the other conduit network 238, ozinated water for sanitizing the carts 23, the system 20 conserves water and is advantageous in this respect.

It will be understood that numerous modifications and substitutions can be had to the aforedescribed embodiment 20 without departing from the spirit of the invention. For example, although the aforedescribed system embodiment 20 has been shown and described as being utilized for washing and sanitizing wheeled grocery store shopping carts 23, the system embodiment 20 is also well-suited for sanitizing and cleaning child strollers for re-use.

Further still, the system 20 can be utilized in conjunction with an electrically-powered means for automatically advancing a cart 23 (of a stack of carts) onto the conveyor 48 following the conveyance of a previously-conveyed cart 23 onto the upper section 29 of the conveyor 48. Such electrically-powered means can involve an electrically-powered wheeled cart, such as is currently available from Dane Technologies of Brooklyn Park, Minn. under the trade designation QuicKart and which is situated behind a stack of carts 23 (like the cart stack illustrated in FIG. 2) positioned in front of the passageway entrance 40 for advancing the stack of carts 23 one at a time onto the upper section 49 of the conveyor 48 upon the occurrence of a predetermined event, such as the receipt of a signal from signal-generating sensors.

For example, such signal-generating sensors can include light sensors which are appropriately mounted on opposite sides of the conveyor 48 for sensing the presence of a cart 23 when the cart reaches a predetermined passageway location (e.g. within the region of the passageway 32 situated beneath the nozzles 154). Upon sensing the presence of the cart 23 at such a predetermined location, the sensor would send appropriate command signals (e.g. wireless signals) to a receiver associated with the electrically-powered means to initiate the incremental advancement of the stack of carts 23 toward the passageway 32. An additional sensor which senses the operative engagement of the wheels of the forwardmost cart 23 (of the cart stack) by the cleats 53 of the conveyor 48 could be responsible for halting the incremental advancement of the stack of carts 23 by the electrically-powered means. It can be appreciated that the utilization of such a system along with such an electrically-powered means for incrementally advancing a stack of carts 23 onto the conveyor 48 would require no manpower for advancing the stack of carts 23 into the passageway 32 and is advantageous in this respect.

Accordingly, the aforedescribed embodiments are intended for the purpose of illustration and not as limitation.

1. A system for sanitizing an item, such as a grocery store shopping cart or a stroller, for re-use, the system comprising:
a source of ozinated water; and
means for directing the ozinated water from the source toward the item so that the surfaces of the item which are exposed to the ozinated water are sanitized thereby.

2. The system as defined in claim 1 wherein the source of ozinated water includes means for generating and injecting ozone into unozinated water to thereby ozinate the water.

3. The system as defined in claim 1 wherein the means for directing includes at least one nozzle through which the ozinated water is directed in a stream toward the item and wherein the at least one nozzle is adapted to impart a swirling motion to the directed water stream.

4. The system as defined in claim 1 wherein the means for directing includes a plurality of stationary nozzles through which the ozinated water is directed toward the item and the system further includes means for conveying the item past the plurality of stationary nozzles so that as the item is conveyed past the stationary nozzles, the item is struck by the ozinated water directed from the nozzles.

5. The system as defined in claim 4 wherein the plurality of stationary nozzles is a first set of nozzles and the system further includes a second set of stationary nozzles through which unozinated water is directed toward the item, and the second set of nozzles is disposed in such a relation to the first set of nozzles so that as the item is conveyed past the stationary sets of nozzles, the item is struck first by unozinated water which is directed from the second set of nozzles and then from the ozinated water which is directed from the first set of nozzles.

6. The system as defined in claim 5 further including means for blowing water from the surfaces of the item after the item has been struck by the unozinated and ozinated water.

7. The system as defined in claim 1 including means for collecting the water after it has been directed toward the item for subsequent use by the directing means.

8. The system as defined in claim 7 wherein there is associated with the source of ozinated water a means for generating and injecting ozone into unozinated water to thereby ozinate the water, and there is associated with the collecting means a means for routing the collected water to the means for generating and injecting where ozone is re-injected into the water.

9. The system as defined in claim 1 wherein the concentration of O₃ within the ozinated water is at least 2 parts per million (ppm).

10. The system as defined in claim 1 further including
means defining a passageway having an entrance through which an item to be sanitized enters the passageway-defining means and an exit through which the item exits the passageway-defining means; and
means for conveying the item through the passageway between the entrance and the exit thereof, and
the water-directing means is adapted to direct the ozinated water from the source toward the item as the item is conveyed between the entrance and the exit of the passageway.

11. The system as defined in claim 10 wherein the water-directing means is also adapted to direct unozinated water toward the item as the item is conveyed between the entrance and the exit of the passageway to dislodge debris from the surfaces of the item before the item is exposed to the ozinated water from the source.

12. The system as defined in claim 11 wherein there is associated with the source of ozinated water a means for generating and injecting ozone into unozinated water to thereby ozinate the water, and the system further includes:
means for collecting the water after it has been directed toward the item; and
means for returning the collected water to the passageway where the water is re-directed toward an item conveyed through the passageway, the means for returning including a first conduit network through which a first portion of the collected water is returned to the passageway and directed toward the item in an unozinated condition and a second conduit network through which a second portion of the collected water is directed to the means for generating and injecting so that ozone is re-injected into the second portion of the collected water so that the second portion of the collected water is returned to the passageway in an ozinated condition.

13. The system as defined in claim 12 wherein the means for conveying is adapted to convey the item through the passageway so that the item is exposed to the unozinated water which is returned to the passageway before it is exposed to the ozinated water which is returned to the passageway.

14. The system as defined in claim 13 further including means for blowing air toward the item following the exposure of the item to unozinated and ozinated water.

15. A system for sanitizing an item, such as a grocery store shopping cart or a stroller, for re-use, the system comprising:
means defining a passageway having an entrance through which an item to be sanitized enters the passageway-defining means and an exit through which the item exits the passageway-defining means;
means for conveying the item through the passageway between the entrance and the exit thereof;
means for directing a stream of unozinated water toward the item as it is conveyed through the passageway to thereby clean the surfaces of the item and a stream of ozinated water toward the item as it is conveyed through the passageway to thereby sanitize the surfaces of the item; and
means for collecting the water after it has been directed toward the item being conveyed through the passageway for purposes of re-directing the collected water toward an item being conveyed through the passageway.

16. The system as defined in claim 15 further including means associated with the passageway-defining means for blowing water from the surfaces of the item following the exposure of the item to the unozinated water and the ozinated water.

17. A method for sanitizing an item, such as a grocery store shopping cart or child stroller, for re-use, the method comprising the steps of:
providing a source of ozinated water; and
directing the ozinated water from the source toward the item to sanitize the surfaces of the item.

18. The method as defined in claim 17 wherein the step of providing a source of ozinated water includes a step of generating and injecting ozone into a quantity of unozinated water to thereby ozinate the water.

19. The method as defined in claim 17 wherein the step of directing is followed by a step of collecting the water after it has been directed toward the item for the purpose of re-directing the collected water toward an item.

20. The method for sanitizing an item, such as a grocery store shopping cart or child stroller, for re-use, the method comprising the steps of:
providing a passageway having an entrance through which an item to be sanitized enters the passageway-defining means and an exit through which the item exits the passageway-defining means;
conveying the item through the passageway between the entrance and the exit thereof;
directing a stream of unozinated water toward the item as it is conveyed through the passageway to thereby clean the surfaces of the item and directing a stream of ozinated water toward the item as it is conveyed through the passageway to thereby sanitize the surfaces of the item; and
collecting the water after it has been directed toward the item being conveyed through the passageway for purposes of re-directing the collected water toward an item being conveyed through the passageway.

21. The method as defined in claim 20 wherein the step of directing the unozinated water and the ozinated water toward the item is followed by a step of:
blowing air toward the surfaces of the item.
